(19) United States
(12) Patent Application Publication
(10) Pub. No.: US 2019/0183894 A1
(43) Pub. Date: Jun. 20, 2019

(54) METHOD OF TREATMENT OF DIABETIC FOOT ULCERS

(71) Applicant: Malesil Research & Technology LLC, Doral, FL (US)

(72) Inventors: John Luis Palacio Barberan, Doral, FL (US); Florencio Jose Gonzalez Bello, Doral, FL (US)

(21) Appl. No.: 16/126,605

(22) Filed: Sep. 10, 2018

(30) Foreign Application Priority Data
Dec. 19, 2017 (MX) MX/A/2017/016930

(51) Int. Cl. A61K 31/519; A61P 17/02

(52) U.S. Cl. CPC A61K 31/519; A61P 17/02

(57) ABSTRACT

A novel method of treating, preventing and maintaining of diabetic foot ulcers is described.

### FIELD OF THE INVENTION

The present invention is related to a method of treating, preventing and maintaining diabetic foot ulcers using sildenafil maleate.

### BACKGROUND OF THE INVENTION

Diabetes is a chronic disease where the body is unable to control blood glucose due to defects in insulin secretion, insulin action or both. Diabetes may lead to a number of complications, resulting from damage exerted by hyperglycemia (blood glucose increase above normal levels) to organs and systems, especially to nerves and blood vessels.

One of the more frequent diabetes complications is diabetic foot, which may be defined as a set of syndromes where the presence of neuropathy, ischemia and infection causes tissue alterations or ulcers secondary to microtraumas.

Diabetic foot ulcers are chronic and complex wounds which use to be a result from one or more simultaneously caused risk factors. A risk factor is peripheral neuropathy, defined as a loss of protective sensitivity such as pain and autonomic dysfunction.

Other risk factors are peripheral arterial disease, increased levels of glycosylated hemoglobin, decrease of visual acuity, record of ulcer or amputation and onychomycosis. External trauma is an instrumental component for ulcer development having as main origin the use of unsuitable footwear.

It is less frequent that peripheral vascular disease is the precipitating event of diabetic foot ulcers; however, it plays an essential role in wound healing and in gangrene development; it is a contributing factor for half of amputations. Although the instrumental or triggering event of ulcer is frequently external trauma, the peripheral vascular disease is the underlying basis of physiopathology of this diabetic foot complication. Even when ulcer pathogenesis is neuropathy, a vascular etiology has been proposed for neuropathy.

Diabetic ulcers show amputation of affected limbs as a main problem; being demonstrated that 85% of lower limb amputations in diabetic patients are preceded by ulcerations, which allow entry of infectious agents, thus causing progressive tissue necrosis with minimum wound healing in the presence of ischemic media.

Amputation rate in diabetic patients is 15 times higher compared to general population. Moreover, it has been noticed that in 58% of patients who have suffered any amputation resulting from diabetic ulcers, there will be a new amputation in their opposite lower limb within the following 3 to 5 years; while mortality within 2 years after the first amputation is of 20 to 50%.

There are several types and grades of diabetic ulcers depending on foot damage. Ulcers are commonly located on foot sole and rarely above foot. There is not any universally accepted classification encompassing assessment criteria of diabetic foot lesions; however, the most common classification is Wagner scale which assesses ulcer depth together with the presence of gangrene and perfusion loss by using six grades (0-5). Table 1, shows the classification according to Wagner scale.

TABLE 1
Classification of diabetic foot ulcers according to
Wagner scale.

| Grade | Lesion | Characteristics |
|---|---|---|
| 0 | None. Foot in | Callus, hallux, mallet toes |
| | risk | |
| 1 | Surface ulcers | Destruction of full skin |
| | | thickness |
| 2 | Deep ulcer | Penetrates skin, fat and |
| | | ligaments without affecting |
| | | bone, infected |
| 3 | Deep ulcers plus | Extensive and deep, secretion, |
| | abscess | bad odor |
| | (osteomyelitis) | |
| 4 | Limited gangrene | Necrosis in a section of the |
| | | foot |
| 5 | Extended gangrene | Whole foot affected, systemic |
| | | effects |

Treatment of diabetic foot ulcers has a main objective of closing the wound; preferably, ulcers should be treated in a preliminary step in order to allow an early cure. The treatment consists of:

a) Optimum diabetes control: keeping or reaching ideal weight; keeping fasting blood glucose levels from 80 to 100 mg/dL and when going to bed from 100 to 140 mg/dL; keeping A1c glycosylated hemoglobin lower than 6%; blood pressure 120/80 mmHg; total cholesterol lower than 200 mg/dL; HDL-cholesterol higher than 35 mg/dL; LDL-cholesterol lower than 100 mg/dL

b) Efficient care of wounds: tissue debridement, inflammation control, humidity equilibrium (selection of optimum dressing)

c) Control of infections: use of antimicrobial agents

d) Strategies to release pressure

e) Restoration of blood flow

In brief, treatment of diabetic foot in addition to metabolic control and associated risk factors, consists of improving circulation and easing ulcer healing within the less possible time, decreasing treatment costs, as well as psychological and social impact.

It has been demonstrated that between 40 to 85% of amputations may be prevented, which enhances the significance of identifying risk factors, search and use of preventive measures, as well as improvement to available treatments.

Nowadays, cilostazol (50 mg and 100 mg) is used as a supplement of diabetic foot treatment, which is a cellular phosphodiesterase III specific inhibitor, having a vasodilator effect, improving circulation; however, it shows side effects such as postural hypotension, sickness, flatulence, palpitations and less frequently, tachyarrhytmias and angina.

Epidermal Growth Factor (EGF) is also used parenterally, intralesionally and around lesion to promote lesion healing and decrease the risk for amputations; however, it has been noticed that patients present pain, burning sensation, chills and fever, derived from application of such medicament.

The prior art has disclosed the use of 5-type phosphodiesterase (PDE5) inhibitors, an enzyme which acts specifically over cyclic guanosine monophosphate (cGMP), such as sildenafil, in treatment of ulcers and wounds, as well as prevention, reduction and reversion of damage to blood vessels caused by diabetes.

Document WO2002015893A2 discloses a method of treatment of chronic venous ulcers, chronic arterial ulcers, chronic decubitus and acute wounds with a cGMP PDE5 inhibitor. WO2011019399A1 describes a method for preventing, reducing or reverting one or more deleterious effects in a blood vessel with a phosphodiesterase inhibitor (PDE), wherein the deleterious effect may be caused by diabetes mellitus.

Document WO2001051042A2 is targeted to the use of cGMP PDE5 inhibitors, particularly, is targeted to the use of sildenafil in treatment of diabetic foot ulcers. On the other hand, WO2002002118A1 discloses a method of treatment and prevention of diabetic foot ulcer formation comprising treating to said patient with an effective amount of a pharmaceutical composition comprising a cGMP PDE5 inhibitor or a derivative or salt thereof. Said document also discloses a method for manufacturing a therapeutic agent of diabetic foot ulcer comprising sildenafil.

However, above mentioned documents, specifically document WO2001051042A2, which might be considered the closest state of the art, does not specify any dose or dosage regime of sildenafil which is useful, efficient and safe for treatment, prevention and maintenance of diabetic foot ulcers.

Moreover, none of the documents makes specific reference to sildenafil maleate, which shows several advantages such as a faster absorption and therefore, a faster action than sildenafil citrate.

### SUMMARY OF THE INVENTION

The present invention describes the treatment, prevention and maintenance of diabetic foot using a 5-type phosphodiesterase (PDE5) inhibitor, specifically the use of sildenafil maleate.

### DETAILED DESCRIPTION OF THE INVENTION

As described in present invention, the “prevention” considers administration of treatment before development of any ulcer, in order to decrease a probability of ulcer development. On the other hand, the term “maintenance” refers to blood flow conservation after disappearance of involved lesions (healing).

As used in present invention, the term “conventional treatment” considers ambulatory wound care, application of antibiotics when needed, use of membranes and healing creams.

“Adverse events” are referred to unfavorable incidents, unexpected facts or undesirable therapeutic accidents and attributable to applied medicament or treatment.

The magnitude of health issue representing the care of diabetes patients, leads to medical staff to maximize available resources and emphasize in prevention of complications of such disease. Ulcers and amputations of lower limbs represent a serious issue in this group of patients. In spite of using all available means nowadays, healing time of foot ulcers in diabetic patients is long, demanding high social, psychological and economic costs, therefore other alternatives need to be searched allowing a faster and more effective cure.

The present invention provides a method of treatment, prevention and maintenance of diabetic foot ulcers, involving administration of sildenafil maleate, which is a 5-phosphodiesterase inhibitor drug and accordingly favors vasodilation. This component, in 10-mg daily dose has demonstrated an improvement in macro and microcirculation, accelerating granulation and healing of ulcers in diabetic foot patients, decreasing the probabilities of amputation of the affected limb, reducing treatment costs and improving patient's quality of life. Its administration is by oral route and sildenafil maleate does not show side effects unlike other medicaments used in such pathologies.

Sildenafil maleate is bioequivalent with sildenafil citrate; however, sildenafil maleate molecule is smaller and has a lower molecular weight, resulting in a faster absorption. Maximum sildenafil maleate concentration is reached after 60 minutes of its oral administration in fasting subjects; when ingested during a high fat content meal, maximum peak may be delayed 60 minutes.

Other advantage of sildenafil maleate is that the drug is widely distributed in tissues (distribution volume in 105-L stationary media); circulates together with its main metabolite N-demethyl sildenafil, mostly bound to plasma proteins.

Surprisingly, the inventors found that treatment with sildenafil maleate not only decreases the average time for ulcer cure in patients with diabetic foot, but further prevents development thereof and maintains blood flow, decreasing the possibility of appearance of future ulcers and amputations.

Specifically, sildenafil maleate exerted a vasodilator action over microcirculation, leading to a more effective granulation and healing.

### EXAMPLES

Examples shown below have the sole purpose of illustrating and demonstrating some embodiments of the invention. Exemplary embodiments shall not be considered limitative of the present invention. As it may be acknowledged by any one skilled in the art, modifications and variations to embodiments herein described may be carried out without altering the essence of the invention.

### Example 1

In order to assess the effect of sildenafil maleate in treatment of diabetic foot ulcers, a 12-week open-label longitudinal study was conducted to compare the conventional treatment and the conventional treatment with simultaneous administration of sildenafil maleate.

Sildenafil Maleate Formulation

A coated tablet containing a low dose of sildenafil maleate was prepared and further being stable for two years (Table 2).

TABLE 2
Composition of coated sildenafil tablets.

| Component | Amount | |
|---|---|---|
| Sildenafil maleate (equivalent to | 13.2 | mg |
| 10 mg base) | | |
| Tribasic calcium phosphate | 40.0 | mg |
| Lactose | 60.2 | mg |
| Corn starch | 40.0 | mg |
| Croscarmellose sodium | 8.0 | mg |
| Microcrystalline cellulose (Avicel | 12.0 | mg |
| pH 101) | | |
| Polyvinylpyrrolidone (PVP K-30) | 10.0 | mg |
| Magnesium stearate | 4.0 | mg |
| Sodium glycolate starch | 12.0 | mg |
| Opadry Pink YS-1-1477 (Red No. 27 D&C | 7.55 | mg |
| lacquer aluminum, blue No. 2 FD&C | | |
| lacquer aluminum, hypromellose, | | |
| titanium dioxide, polyethylene | | |
| glycol, polysorbate 80) | | |
| Methylene chloride | 0.02096 | mL |

Sampling

Diabetic patients with grade 2 and grade 3 ulcers were selected, according to Wagner classification and complying with the following inclusion and exclusion criteria:

Inclusion Criteria:
Male or female patients with type-2 Diabetes Mellitus
Age younger than 80 years
Grade-2 or -3 foot or leg ulcer, of any origin according to Wagner classification, without compromising tendons nor bone, with a progression time not higher than 3 months.
Presence or absence of localized infection
Without peripheral vascular obstruction

Exclusion Criteria
Type-1 Diabetes Mellitus patients
Grade-4 foot or leg ulcer according to Wagner classification or foot or knuckle necrosis
Peripheral vascular obstruction (ankle/arm index lower than 0.3)
Patients with creatinine higher than 1.2 mg/dL or blood urea nitrogen (BUN) higher than 50
Transaminases 2 times above baseline
Microalbuminuria higher than 100

Study Variables

During patient recruitment, the variables reported in Table 3 were studied.

TABLE 3
Study variables

| SPECIFIC VARIABLE | CATEGORY-SCALE |
|---|---|
| Ulcer presence | Yes/No |
| Ulcer location | Forefoot, heel, toes, leg, etc. |
| Wagner classification | 1, 2, 3, 4 |
| Ulcer size | Centimeters |
| Progression time | Days |
| Previous Treatment | Yes/No; time; type |
| Infection presence | Yes/No |
| Use of antibiotics | Yes/No; type; time |
| Healing | Yes/No |
| Age | Years |
| Gender | F/M |
| Time with diabetes | Years |
| DB Treatment | Yes/No; type |
| Degree of metabolic | Good: HbA1c lower than 7%; bad: |
| control | control HbA1c higher than or |
| | equivalent to 7% |
| Education | Elementary, high school, college, |
| | postgraduate, illiterate. |
| Income level | High, medium, higher low, lower low |
| Vascular assessment | Doppler of affected limb, presence |
| | of obstruction; obstruction location |
| Assessment of | Neurological assessment LIFE |
| amputation risk | protocol; diabetic neuropathy |

Protocol

Patients who meet inclusion and exclusion criteria, who voluntarily accept to participate in the study and further signing a written consent, are randomly assigned to two study groups:

Group I: Conventional treatment

Group II: Conventional treatment with simultaneous administration of sildenafil maleate.

Patients are required to perform wound treatment and to register lesion progression.

Visit 1
Signature of written consent
Physical examination
Petition of clinical lab examinations: complete blood count, glycosilated hemoglobin (HbA1c), total colesterol, total lipids, triglycerides, HDL-colesterol, urea, creatinine, microalbumina, aspartate aminotransferase (TGO), alanine aminotransferase (TGP), reactive, ultra-sensitive C-protein (PCR-u), urine test, pre and postprandial glycemia, lesion culture when required.
Conventional healing of lesion
Arterial Doppler request of affected limb
Provide treatment with antibiotics when necessary

Visit 2 (1 to 3 Days after Visit 1)
Interpretation of lab examination and Doppler study
Clinical/physical lesion examination
Patient inclusion/exclusion decision into the study
Random assignment of study groups
Registration of lesion characteristics (lesion photographs)
Assessment of metabolic control

Visit 3 (1 to 3 Days after Visit 2)
Registration of lesion progression (lesion photographs)
Assessment of patient's metabolic control
Delivery of corresponding medicaments (antibiotics and sildenafil maleate as applicable)
Registration de adverse events

Visit 4 Until Week 12 of Study (2 to 3 Times Per Week)
Registration of lesion progression (lesion photographs)
Registration de adverse events
Assessment of metabolic control

Visit 5 (1 to 5 Days after Final Visit)
Registration of lesion progression (lesion photographs)
Registration of adverse events
Compliance with study medication intake
Education for future lesion prevention

Foot Assessment

Foot assessment of diabetic patient includes 4 components: vascular, neuropathic, orthopedic and infectious components; the presence of these components may define the prognosis for lesion progression. Assessment is carried out based on criteria described in Table 4.

TABLE 4
Foot Assessment

| Component | Assessment |
|---|---|
| Neuropathy | Head of metatarsal bone 1, 2, 3 and 5 and |
| | first toe of foot are assessed with a 10 g |
| | Semmens Weinsten monofilament. It is |
| | positively determined if patient does not |
| | perceive the monofilament in more than |
| | one site. |
| Vascular | Femoral, popliteal, pedal and tibial |
| | posterior pulses; forefinger, ankle, arm |
| | are assessed by palpation; Doppler of |
| | lower limbs. |
| Orthopedic | Alteration of foot shape producing |
| | difficulty for using standard footwear |
| | (unmodified); it may include flat foot or |
| | high-arched foot, mallet toes, bunions, |
| | previous amputations. |
| Infectious | Presence of infection signs which |
| | aggravates ulcer progression and requires |
| | the use of antibiotics; may be located or |
| | systemic. |
| History of | Review of medical records; patient is |
| previous ulcer | asked about. Previous lesions increase |
| and background | the risk of complications. |
| Disability | Physical: the patient cannot reach |
| | his/her feet. |
| | Visual: the patient does not see his/her |
| | feet sufficiently well in a safe manner |
| | to cut his/her nails. |

Assessment of Amputation Risk

The risk of amputation is based on the presence of any neuropathic, vascular or orthopedic complication impeding or retarding a normal healing.

Low risk is considered when detected at least one pulse on each foot, there is no neuropathy, no deformity, no disability and absence of ulcer records.

On the other hand, when there is an ulcer or amputation record, absence of pulses and neuropathy or one of those above with deformity, a high risk is present.

Comorbidity

Comorbidity is a coexistence of two or more pathologies. On this regard, a number of epidemiological studies show that diabetes is associated with arterial hypertension (HTA), diabetic neuropathy, dyslipidemia (DISL), hypothyroidism, diabetic retinopathy, diabetic nephropathy, cardiopathy, depression, cerebral-vascular disease (ECV), and similar diseases. Comorbidity was determined based on medical records and clinical studies.

Wound Treatment and Follow Up

Wound treatment applied to all patients during each visit consists of cleaning with 9% saline and distilled water, using sterile material; ointment MEBO® (Moist exposed Burn Treatment) was locally applied and dressing with sterile gauze when needed.

Study medication was delivered in accordance with the previously established protocol; compliance with medication intake and presence of adverse events was verified. Medication was suspended in case of showing any adverse effect, even a mild event associated with study medicament (sildenafil maleate).

Results

Table 5 shows general results of patients belonging to study Group I (conventional treatment), while Table 6 shows results obtained from a study conducted on patients belonging to Group II (conventional treatment with simultaneous administration of sildenafil maleate).

100% of patients in Group II (conventional treatment with simultaneous administration of sildenafil maleate) obtained a satisfactory result (lesion healing); while only 85% of patients in Group I (conventional treatment) obtained a satisfactory result.

TABLE 5
General results of patients belonging to study Group I (Conventional treatment)

| | | | | | | | | | | | WEEKS |
|---|---|---|---|---|---|---|---|---|---|---|---|
| | | | | | | | | | | | OF |
| PATIENT | | | YEARS | | METABOLIC | | AFFECTED | WAGNER | AREA | INFEC- | TREAT- |
| CODE | SEX | AGE | W/DIABETES | COMORBIDITY | CONTROL | FOOT | ZONE | CLASSIF | (cm²) | TION | MENT |
| V-I-01S | F | 80 | 30 | HTA-DYSL-EAC | HG ORAL | L | MALEOLAR | W-2 | 4 | YES | 15 |
| V-I-02S | F | 61 | 42 | HTA-EAC | INSULIN | L | HEEL | W-3 | 20 | YES | 16 |
| V-I-05S | F | 78 | 8 | HTA-EAC | INSULIN | R | PLANTAR | W-3 | 3 | YES | 17 |
| V-I-06S | M | 45 | 6 | HTA-DYSL-EAC | HG ORAL | R | AMP BED | W-3 | 8 | NO | 12 |
| V-I-07S | M | 44 | 20 | HTA-DYSL | INSULIN | L | AMP BED | W-3 | 3 | YES | |
| V-I-08S | M | 48 | 5 | HTA-DYSL-AEV | HG ORAL | R | EXTERNAL | W-3 | 10 | YES | 12 |
| V-I-09S | M | 55 | 19 | HTA-DYSL-EAC | HG ORAL | R | 1^{ST} TOE | W-2 | 1 | NO | 13 |
| V-I-10S | F | 60 | 10 | HTA-DYSL | HG ORAL | L | DORSOLATE | W-2 | 15 | YES | 15 |
| V-I-11S | M | 82 | 32 | HTA-DYSL | HG ORAL | R | AMP BED | W-3 | 2 | YES | 15 |
| V-I-12S | F | 58 | 10 | HTA-DYSL | HG ORAL | R | 2ND, 3^{RD} TOE | W-2 | 1 | YES | 4 |
| V-I-13S | M | 73 | 1 | HTA | HG ORAL | L | AMP BED | W-3 | 1 | YES | |
| M-1-M | F | 58 | 10 | HTA | INSULIN | R | KNUCKLE | W-2 | 2 | YES | 15 |
| M-2-M | M | 60 | 5 | HTA | HG | R | HEEL | W-2 | 2 | NO | 13 |
| M-3-M | M | 52 | 18 | HTA | INSULIN | R | PLANTAR R | W-2 | 2 | YES | 7 |
| M-4-M | M | 55 | 5 | OBESITY | HG | L | 1ER KNUCKLE | W-2 | 2 | YES | 7 |
| M-5-M | F | 54 | 3 | DYSL | HG | L | HEEL | W-2 | 2 | YES | 8 |
| M-6-M | F | 73 | 10 | HTA | INSULIN | R | LEG INT | W-2 | 2 | YES | 8 |
| | | | | | | | FACE | | | | |
| M-7-M | M | 65 | 4 | DYSL | HP | L | PLANTAR | W-2 | 2 | NO | 16 |
| M-8-M | F | 61 | 5 | OBESITY | HG | R | LEG INT | W-2 | 4 | YES | 7 |
| | | | | | | | FACE | | | | |
| M-9-M | M | 58 | 4 | DYSL | HG | L | 2^{ND} TOE | W-2 | 2 | YES | |
| M-10-M | M | 56 | 10 | HTA | INSULIN/HG | L | PLANTAR | W-2 | 2 | NO | 8 |
| M-11-M | M | 71 | 12 | HTA | HG | R | 1^{ST} TOE | W-2 | 2 | NO | 3 |
| M-12-M | M | 47 | 10 | DYSL | HG | R | KNUCKLE | W-2 | 2 | YES | 7 |
| M-13-M | M | 63 | 4 | HTA | HG | R | REAR FACE | W-2 | 4 | YES | 11 |
| M-14-M | F | 58 | 13 | HTA | INSULIN | L | HEEL | W-3 | 10 | YES | |
| M-15-M | M | 49 | 4 | HTA | HG | L | PLANTAR | W-2 | 2 | YES | 7 |

TABLE 6
General results of patients belonging to study Group II (Conventional treatment and
sildenafil maleate)

| | | | | | | | | | | | WEEKS |
|---|---|---|---|---|---|---|---|---|---|---|---|
| | | | | | | | | | | | OF |
| PATIENT | | | YEARS | | METABOLIC | | AFFECTED | WAGNER | AREA | INFEC- | TREAT- |
| CODE | SEX | AGE | W/DIABETES | COMORBIDITY | CONTROL | FOOT | ZONE | CLASSIF | (cm²) | TION | MENT |
| V-I-01M | M | 61 | 30 | HTA-ECV-DYSL | INSULIN | L | AMP BED | W-2 | 2 | YES | 7 |
| V-I-02M | F | 58 | 10 | HTA-ICC | ORAL HG | R | PLANTAR | W-2 | 4 | YES | 7 |
| V-I-03M | M | 47 | 3 | HTA-DYSL-OB | ORAL HG | R | FOREFOOT | W-2 | 20 | YES | 15 |
| V-I-04M | M | 85 | 40 | HTA | ORAL HG | R | METATAR | W-2 | 6 | YES | 11 |
| V-I-05M | F | 45 | 2 | HTA-EAC | ORAL HG | R | AMP BED | W-2 | 5 | NO | 6 |
| V-I-06M | F | 61 | 42 | HTA-EAC | INSULIN | R | HEEL | W-2 | 8 | NO | 7 |
| V-I-07M | M | 55 | 10 | HTA | INSULIN | L | AMP BED | W-2 | 6 | YES | 4 |
| V-I-08M | M | 60 | 21 | HTA | ORAL HG | L | PLANTAR | W-2 | 1 | NO | 7 |
| V-I-09M | M | 53 | 15 | EAC-DYSL | ORAL HG | L | HEEL-FOOT | W-3 | 15 | YES | 13 |
| V-I-10M | M | 63 | 2 | DYSL | ORAL HG | R | PLANTAR | W-3 | 20 | YES | 12 |
| V-I-12M | F | 73 | 7 | HTA | ORAL HG | R | AMP BED | W-2 | 6 | NO | 9 |
| V-I-13M | M | 57 | 20 | EAC-DYSL | ORAL HG | L | TAL-PL-DOR | W-3 | 20 | YES | 11 |
| V-I-14M | M | 49 | 10 | DYSL | INSULIN | L | AMP BED | W-2 | 2 | YES | 9 |
| I-10-S | M | 57 | 15 | HTA | HG | R | KNUCKLE | W-2 | 2 | YES | 4 |
| I-11-S | M | 58 | 10 | HTA | HG | L | EXT FACE | W-2 | 3 | YES | 6 |
| I-12-S | M | 39 | 2 | HTA | INSULIN | R | DORSAL | W-2 | 2 | YES | 3 |
| I-13-S | M | 51 | 5 | OBESITY | INSULIN/HG | R | PLANTAR | W-2 | 1 | NO | 3 |
| I-14-S | M | 57 | 12 | HTA-DYSL | INSULIN | L | 3^{RD} TOE | W-3 | 1 | YES | 7 |
| I-16-S | M | 43 | 3 | HTA | HG | R | AMP BED | W-2 | 4 | YES | 4 |
| I-18-S | F | 63 | 20 | OBESITY | INSULIN/HG | R | PLANTAR | W-3 | 5 | YES | 10 |
| I-19-S | F | 43 | 12 | HTA | INSULIN/HG | R | PLANTAR | W-2 | 3 | YES | 8 |
| I-20-S | F | 53 | 7 | HTA-OBESITY | HG | R | PLANTAR | W-2 | 4 | YES | 7 |
| I-21-S | F | 76 | 11 | HTA | INSULIN | L | INT FACE | W-3 | 15 | YES | 16 |
| I-22-S | M | 56 | 5 | HTA | HG | L | KNUCKLE | W-2 | 1 | YES | 11 |
| I-23-S | M | 53 | 12 | HTA | INSULIN/HG | R | PLANTAR | W-2 | 4 | YES | 3 |
| I-24-S | M | 79 | 7 | HTA | INSULIN | R | KNUCKLE | W-2 | 1 | YES | 4 |

Healing Time

It was surprisingly found that regardless of the ulcer grade according to Wagner scale and the lesion area, patients treated with sildenafil maleate decreased the recovery time by about 20 days.

As appreciated from Tables 7 and 8, patients who showed grade 2 ulcers and treated with sildenafil maleate, though having 296 more of average area of lesion demonstrated a 20 days lower healing than the group with conventional treatment. In case of patients with grade 3 ulcer, the result is better than the group treated with sildenafil maleate, although such group duplicates the average area of lesion of the group with conventional treatment.

TABLE 7
Area of lesions

| | Average of | Average of |
|---|---|---|
| Ulcer grade as per | lesion area | lesion area |
| Wagner scale | Group I (cm²) | Group II (cm²) |
| 2 | 2.94 | 3.80 |
| 3 | 4.88 | 12.67 |

TABLE 8
Healing time for grade 2 and 3 ulcers according to
Wagner scale

| Ulcer grade | Average healing | |
|---|---|---|
| according to Wagner | time | Average healing time |
| scale | (Group I) | (Group II) |
| 2 | 67 days | 47 days |
| 3 | 101 days | 81 days |
| 2 and 3 | 75 days | 55 days |

A total recovery was observed in the group of patients treated with sildenafil maleate and who further has previous amputations. However, 15% of patients with conventional treatment suffered amputations due to development of existing lesions and appearance of new ulcers.

Moreover, it was noticed that patients in Group II, whose treatment concluded previous to the 12 weeks provided in present study, kept an improvement in blood flow and generally in health status of lower limbs.

Adverse Events

Although sildenafil maleate is a vasodilator agent, patients belonging to Group II (treated with sildenafil maleate) and further presenting arterial hypertension (HTA), did not experience adverse events.

1. A method of treating, preventing and maintaining of diabetic foot ulcers comprising administering to a patient sildenafil maleate.

2. The method according to claim 1, wherein the administration of the sildenafil maleate accelerates granulation and healing of diabetic foot ulcers.

3. The method according to claim 1, wherein the administration of the sildenafil maleate prevents appearance of future ulcers.

4. The method according to claim 1, wherein the administration of the sildenafil maleate prevents the amputation associated with diabetic foot.

5. The method according to claim 1, wherein the method comprises administering 10 mg of sildenafil maleate.

6. The method according to claim 1, wherein the method comprises administering sildenafil maleate once a day.

7. The method according to claim 1, wherein the method comprises administering sildenafil maleate orally as a tablet.

8. The method according to claim 1, wherein the method comprises administering sildenafil maleate simultaneously to a conventional treatment of diabetic foot ulcers.
